(19) United States
(12) Patent Application Publication
(10) Pub. No.: US 2018/0021373 A1
(43) Pub. Date: Jan. 25, 2018

(54) Ruthenium and Indium Binding to Gastrins

(71) Applicant: EUPHARMA PTY LTD, Northbridge (AU)

(72) Inventor: Malvin EUTICK, Northbridge (AU)

(73) Assignee: Eupharma Pty Ltd, Northbridge (AU)

(21) Appl. No.: 15/548,322

(22) PCT Filed: Feb. 4, 2016
(86) PCT No.: PCT/AU2016/050063

§ 371 Date: Aug. 2, 2017

(30) Foreign Application Priority Data
Feb. 4, 2015 (AU) 2015900343

(51) Int. Cl. A61K 33/24; G01N 33/574; C07K 14/72; A61K 51/08; A61K 38/22

(52) U.S. Cl. CPC A61K 33/24; A61K 51/08; A61K 38/2207; C07K 14/72; G01N 33/574

(57) ABSTRACT

The present invention relies on the hitherto unrealised high affinity binding of ruthenium and indium to gastrins. Particularly, these metals can be bound directly to the gastrins under mild conditions and so can be used in treatment of conditions associated with elevated gastrin levels and in detection of tumours and other conditions where CCK receptors are over expressed.

### FIELD OF THE INVENTION

The invention relates to use of the high affinity binding of ruthenium or indium to gastrins in methods of treatment and detection and also to the complexes formed by said gastrins and the bound ruthenium or indium. Particularly, although not exclusively, the invention relates to the treatment or diagnosis of conditions associated with elevated concentrations of gastrins or gastrin receptors through the use of ruthenium and indium binding.

### BACKGROUND TO THE INVENTION

Any reference to background art herein is not to be construed as an admission that such art constitutes common general knowledge in Australia or elsewhere.

Gastrin, or Gamide, is a well-known gut peptide hormone, which was identified originally as a stimulant of gastric acid secretion (Dockray, 2001). It is produced principally by the G cells of the gastric antrum and, to a variable extent in the upper small intestine, with much lower amounts in the colon and pancreas. The related hormone cholecystokinin (CCK), which is responsible for stimulation of pancreatic enzyme secretion via its binding to the CCK1 receptor, has the same C-terminal tetrapeptide amide as gastrin. Gastric acid secretion stimulated by Gamide is mediated by the cholecystokinin-2 (CCK-2) receptor.

The human CCK-2R (447 amino acids) shares 46% identity in amino acid sequence with the human cholecystokinin1 receptor (CCK1R) (428 amino acids), and both are typical 7-transmembrane domain receptors. The minimum sequence requirement for high-affinity binding to both the CCK1R and the CCK2R is the amidated C-terminal tetrapeptide Trp-Met-Asp-Phe. The two receptors may be readily distinguished by the fact that the CCK1R binds sulfated CCK8 with 500-fold greater affinity than unsulfated CCK8, whereas the difference for the CCK2R is only 10-fold.

The production of the active forms of gastrin in humans starts with the initial translation product of the gastrin gene, which is a large precursor molecule, preprogastrin (101 amino acids). Preprogastrin is converted to progastrin (80 amino acids) by cleavage of the N-terminal signal peptide, and progastrin is processed further within secretory vesicles by endo- and carboxy-peptidases to yield glycine-extended gastrins. The C-terminus of glycine-extended gastrin (Ggly) is then amidated by peptidyl α-amidating mono-oxygenase, and further proteolytic cleavage results in mature amidated gastrin (ZGPWLEEEEEAYGWMDFamide, Gamide). In healthy humans progastrin and the glycine-extended gastrins comprise less than 10% of circulating gastrins.

Gamide/Gastrin is an important growth factor for the gastric epithelium, and is known to stimulate proliferation of the ECL cells of the stomach and proximal small intestine, and gastric parietal cell migration. This proliferative effect can result in carcinoid tumour formation secondary to prolonged hypergastrinaemia in conditions such as Zollinger-Ellison syndrome. A recent report that 80% of human gastric adenocarcinomas co-express Gamide and the CCK2 receptor suggests that many gastric cancers may utilise Gamide as an autocrine growth factor (Goetze J P, 2013).

The precursors, progastrin and its glycine-extended derivatives (Ggly), have previously been regarded as physiologically inactive. However, data has been accumulating to suggest that these gastrin precursors such as Ggly stimulate proliferation in several cancer cell lines (Aly A, 2004) (Ferrand A, 2006).

The major physiological role of progastrin and Ggly is in the colon as progastrin and Ggly stimulate proliferation of a colonic cell line in vitro (Hollande F, 1997) and of the normal mucosa in vivo (Wang T C, 1996) (Koh T J, 1999). Such non-amidated gastrins also act as growth factors in colorectal cancers (Aly A, 2004). Wang et al. (Wang T C, 1996) demonstrated the growth effects of the precursor non-amidated gastrins in normal colonic tissue in vivo. For example, infusion of Ggly into gastrin-deficient mice increased the colonic proliferative index by 80%, but infusion of gastrin/Gamide had no effect on the proliferative index in the colon. Transgenic mice over-expressing human progastrin in the liver have high concentrations of circulating progastrin, but normal gastrin/Gamide concentrations. These mice have thickened colonic mucosa, with deeper crypts and an increased proliferative index in both proximal and distal colon compared to wild type mice. Similar results have been reported for transgenic mice over-expressing Ggly.

Upregulation of gastrin gene expression may contribute to colorectal cancer (CRC) tumorigenesis. Increased concentrations of incompletely processed gastrins have been shown to be present in colonic polyps and adenocarcinomas and in the circulation of CRC patients, and to exert mitogenic effects on normal colorectal mucosa, and in CRC cell lines in vitro and in vivo. Gastrins also have proangiogenic properties. Tubule formation by human vascular endothelial cells is enhanced by both Gamide and Ggly. Treatment of human CRC cell lines with Ggly has been shown to stimulate expression of the major angiogenic factor vascular endothelial growth factor (VEGF), and colons from transgenic mice overexpressing Ggly have been demonstrated to exhibit higher levels of VEGF expression and greater microvessel density compared to control mice.

While these gastrin precursor molecules are implicated in the development of colorectal cancer, the CCK-2 receptor is also implicated in a separate group of cancers where the tumour types have been shown to express the CCK-2 receptor frequently. In particular Reubi and co-workers (Reubi J C, 1997) have demonstrated that more than 90% of medullary thyroid carcinomas and ovarian stromal carcinomas, and more than 50% of astrocytomas and small cell lung carcinomas are CCK-2 receptor positive (see below table).

| Tumour Type | Frequency* | CCK2R + ve/Total (32) | |
|---|---|---|---|
| Medullary thyroid ca | 4% | 22/24 | (92%) |
| Astrocytoma | 50% | 11/17 | (65%) |
| Small cell lung ca | 15% | 8/14 | (57%) |
| Ovarian stromal ca | 8% | 3/3 | (100%) |

*% of all tumours arising from that tissue (e.g. astrocytomas as % of all brain tumours).

There has been interest in the use of metal chelate-conjugated gastrin derivatives for the diagnosis of CCK2R-positive tumours (Roosenburg S, 2011). For example, the chelating group 1,4,7,10-tetraazacyclodecane-1,4,7,10-tetraacetic acid (DOTA) has been coupled to minigastrin₁₁ (D-Glu-Ala-Tyr-Gly-Trp-Met-Asp-Phe-NH₂) and the minigastrin radiolabelled with ¹¹¹In or ⁶⁸Ga (von Guggenberg E, 2012). One of the disadvantages of this approach is that incorporation of the metal ion requires harsh conditions (pH 4.5, 98° C., 15 min), which has been shown to potentially result in oxidative damage or modification to the peptide.

U.S. Pat. No. 6,180,082 addresses the use of receptor dependent radiolabelled compounds and their accumulation in tumour tissues for both diagnosis and treatment. Cholecystokinin (CCK) and somatostatin are disclosed as suitable receptor-dependent peptides. While CCK is a related peptide to gastrin it lacks the gastrin pentaglutamate sequence. A range of already available metal-chelate-peptide complexes, such as ¹¹¹In-Pentetreotide and various DOTA and DPTA derivatives (organic chelating groups) are specified to bind the radioactive metals. It is claimed that the delivery of such peptide chelates with bound metal radionuclides over a longer infusion period provides advantages in improved accumulation with the tumour tissues than through bolus injection.

Roosenburg S, (2011) describes the use of, amongst others, CCK and gastrin peptides in the visualisation, through the use of radiolabeled compounds, of cancers expressing CCK-2R. The conjugation of a chelator to the peptide is discussed as an essential step in allowing radiolabeling with a radioactive metal but the drawbacks of some existing peptide-chelate-metal complexes are described. Particularly, certain complexes require steps to form the product, such as heating, which potentially damage the peptide structure, and others have been shown to demonstrate poor in vivo stability and/or undesirable renal accumulation leading to kidney damage. Once again, DOTA, DTPA and also HYNIC, are described as organic metal chelating groups. Demogastrins 1 and 2, minigastrin analogues wherein a tetramine chain functions as the metal chelator group conjugated to the peptide sequence, are also discussed in relation to technetium (Tc) binding as the metal radionuclide.

Similarly, Fani M 2012, has shown that radiolabeled peptides can be valuable biological tools for tumour receptor imaging and targeted radionuclide therapy. CCK and gastrin peptide analogues are discussed and Tc-demogastrin as well as In-DOTA minigastrins are described as commonly used candidates with one of the main steps in developing such a suitable radiolabeled peptide being described as covalent attachment of a chelating agent or a prosthetic group which will bind the metal. The development of suitable chelating groups is therefore an important component in the art of generating receptor targeted radiolabeled peptides which have appropriate efficacy and in vivo stability.

Tc and In have been used as the radionuclide for attachment to the chelate group to the peptide complex but it will be appreciated that metals generally have been used in a variety of anticancer agents for some time. WO 2007/101997 describes the development of new ruthenium (Ru) sandwich complexes for use in the treatment of cancer. It is stated that related Ru sandwich complexes have been shown to bind DNA directly through the hydrolysis of a halo atom on the complex thereby activating the compound for intercalation and binding. The compounds of WO 2007/101997 act differently in that the moiety corresponding to the halo atom of the earlier compounds does not hydrolyse readily and so it is supposed that it is the intact sandwich complex which is itself the active species. The Ru atom in such species is fully complexed and so cannot itself directly bind to another group. Thus, Ru sandwich complexes largely remain intact and follow a direct mode of action via DNA binding, whatever the activated complex itself may be.

Gastrins bind two ferric ions, the first to Glu7 and the second to Glu8 and Glu9. Ferric ions are essential for the biological activity of non-amidated forms of the peptide, such as Ggly, as a stimulant of cell proliferation and migration. Thus, either the substitution Glu7 to Ala, or treatment with the iron chelator desferrioxamine, completely blocks the biological activity of Ggly. In contrast, ferric ions are not required for the biological activity of Gamide. Bi³⁺ ions, by competing for the ferric ion binding site of Ggly, block biological activity in vitro and in the normal colorectal mucosa in both mice and rats in vivo.

The Ggly metal binding site is highly selective, as Baldwin has shown that Ggly does not bind Co(II), Cu(II), Mn(II), or Cr(III) ions, as detected by measurement of Ggly fluorescence in the presence of added metal ions (Baldwin G S, 2001). It has also been shown that progastrin does not bind Ca(II), Co(II), Cu(II), Fe(II), Mn(II) or Zn(II), or Al(III), Cr(III) or Eu(III) ions, as detected by competition with radioactive ⁵⁹Fe³⁺ ions (Baldwin G, 2004).

WO 2004/089976 discloses that the natural ligand for the Ggly receptor may be the complex formed when the ferric ions bind to Ggly, rather than Ggly itself. Based on this and the knowledge that Ggly has its action independently of the CCK-2 receptors, where Gamide has its effect, they proposed a selective treatment based on the use of trivalent metal ions to block the biological activity of Ggly. Data is provided for Bi³⁺ and Ga³⁺ and it is stated that Co²⁺, Cr³⁺, Cu²⁺ and Mn²⁺ ions were shown to not quench fluorescence and nor did 20 equivalents of Al³⁺ cause any significant shift in Ggly NMR signals, thereby confirming the unpredictable nature of the Ggly metal ion binding, even amongst the trivalent ion class. The data discussed indicates that Ggly binds two Bi³⁺ ions at pH 4 with an affinity of 5.8 uM which is ten-fold lower than for ferric ions.

### SUMMARY OF INVENTION

The present invention is predicated, at least in part, on the surprising finding that ruthenium and/or indium metal ions will bind to gastrins with binding constants which are orders of magnitude below those experimentally determined for Fe³⁺ ions, and even for Bi³⁺ and Ga³⁺ ions. It has been found that both ruthenium and indium will bind to both amidated and non-amidated gastrins with similarly efficacious binding. Given the highly unpredictable nature of the binding of metal ions by gastrins, the fact that ruthenium and indium bind, and the extent of their significantly stronger binding, could not have been predicted from the prior art.

The high affinity binding allows the ruthenium or indium to be bound directly to the gastrin under relatively mild conditions thereby allowing for use of the gastrin with bound ruthenium or indium in methods of treatment or as a tool for the exploration and identification of non-amidated gastrin receptors. This provides a significant advantage over prior art peptide chelate compounds wherein often harsh conditions must be used to introduce the radiolabel to the chelated peptide. Particularly, the binding of ruthenium or indium to the gastrin allows for the treatment of a variety of conditions associated with or caused by amidated or non-amidated gastrins.

The complexes formed from ruthenium or indium and the gastrins, when radiolabelled ruthenium or indium are used, can be employed as probes to identify receptors to which the gastrins bind and hence find use as diagnostic tools in identifying certain conditions. Thus, it will be appreciated that, employing the approach of the present invention, the high affinity binding of ruthenium and indium means there is no need for the conjugation of a metal chelate to the gastrin peptide as the metals will bind directly to the pentaglutamate sequence. This provides for ease of synthesis of the probe/therapeutic and minimises damage to the gastrin and reduces the concerns over in vivo stability.

Further features and advantages of the present invention will become apparent from the following detailed description.

### BRIEF DESCRIPTION OF THE DRAWINGS

In order that the invention may be readily understood and put into practical effect, preferred embodiments will now be described by way of example with reference to the accompanying figures wherein:

FIG. 1 is a representation of the 2 site model of binding of gastrins;

FIG. 2 is the XAS K-edge near edge spectrum of Fe^{III}₂Ggly;

FIG. 3 A-H are a series of EXAFS spectra (A, C, E and G) and their corresponding Fourier transforms (B, D, F and H) of complexes of the metals tested with Ggly wherein A and B were obtained for Fe³⁺ ions, B and C for Ga³⁺ ions, and E and F for In³⁺, and G and H for Ru³⁺;

FIG. 4A is a proposed structural model for Fe^{III}₂Ggly;

FIG. 4B is a proposed structural model for Ru^{III}₂Ggly;

FIG. 5 is a graphical representation of the change in absorption of the gastrins Gamide and Ggly recorded upon addition of Ga³⁺ ions;

FIG. 6 is a graphical representation of the change in absorption of the gastrins Gamide and Ggly recorded upon addition of Fe³⁺ ions in the presence of various concentrations of In³⁺ ions;

FIG. 7 is a graphical representation of the change in absorption of the gastrins Gamide and Ggly recorded upon addition of Fe³⁺ ions in the presence of various concentrations of Ru³⁺ ions; and

FIG. 8 represents purification of a Ru¹⁰⁶-Gamide complex wherein A represents the radioactivity observed in various fractions after Sep-Pak separation and B represents further purification of the complex by anion exchange HPLC wherein the radioactivity in each 1 mL fraction is indicated by the bars.

### DETAILED DESCRIPTION

### Definitions

Unless defined otherwise, all technical and scientific terms used herein have the same meaning as would be commonly understood by those of ordinary skill in the art to which this invention belongs.

As used herein, the terms “gastrin” and “gastrins” refer to progastrin as well as amidated (Gamide) and non-amidated gastrins (glycine-extended gastrins). In one embodiment, the terms may exclude gastrins and gastrin-like peptides, which do not contain some or all of the residues of the characteristic pentaglutamate sequence.

As used herein, the term “amidated gastrins” refers to gastrins which are derived from progastrin and which contain its pentaglutamate sequence and which, additionally, comprise the sequence trp-met-asp-phenylalanine-amide at the C-terminus. Examples of such amidated gastrins include, but are not necessarily limited to, amidated gastrin₃₄ and amidated gastrin₁₇ (Gamide).

As used herein, the term “non-amidated gastrins” refers to gastrins which are or are derived from progastrin and which comprise its pentaglutamate sequence but which do not comprise a phenylalanine-amide at the C-terminus and includes within its scope glycine-extended gastrins. Examples of such non-amidated gastrins include, but are not necessarily limited to, progastrin itself as well as glycine-extended gastrin₁₇ (Ggly) and N- and C-terminally extended forms of glycine-extended gastrin as well as shorter peptides derived from the progastrin sequence between residues 55 and 72.

As used herein the terms “CCK receptor”, “CCK-1 receptor” and “CCK-2 receptor” refer to the cholecystokinin receptor family generally or the specified subtype.

According to a first aspect of the invention, there is provided a method of treatment or prophylaxis of a condition in a patient associated with elevated concentrations of a non-amidated gastrin including the step of administering an effective amount of a metal selected from indium and ruthenium to the patient.

The metal is administered to the patient such that the metal binds to the ferric binding site of the non-amidated gastrin.

In one embodiment, the method further includes the step of selecting a patient in need of such treatment, including in need of the administration of a metal selected from ruthenium and indium to bind to the non-amidated gastrin.

Amidated and non-amidated gastrins elicit different biological effects via distinct receptors in different tissues. Amidated gastrin (Gamide) stimulates gastric acid secretion and the development of gastric carcinoids, whereas the precursor, glycine-extended Gastrin stimulates proliferation of the colonic mucosa and the development of colorectal cancers.

As discussed above, Baldwin (Baldwin, 2001) has shown that Glycine-extended gastrin (Ggly) binds two ferric ions with high affinity. Investigations of the identity of the iron ligands, their binding sites and the role of ferric ions in biological activity have determined that Glu7 is critical for binding the first ferric ion, and that Glu8 and Glu9 are involved in binding the second ferric ion. The complete lack of activity of a Ggly mutant in which Glu7 was replaced by Ala (GglyE7A), and the inhibition of Ggly activity by the iron chelator desferrioxamine (DFO), indicate that ferric ion binding is essential for the biological activity of Ggly.

The results presented in the experimental section demonstrate that indium and ruthenium ions will bind to non-amidated gastrins with unexpectedly high affinities. In³⁺ has been found to have a K_{d} value of 2.1×10⁻¹³ M for the first iron binding site of Ggly versus 5.7×10⁻⁹ M for Fe³⁺. Ru³⁺ has been found to have a K_{d} value of 5.3×10⁻¹⁵ M for the first iron binding site of Ggly. The affinity for In is therefore orders of magnitude higher. The binding affinities of a range of metals has been tested with highly variable results (other group 15 ions such as arsenic and antimony did not show high affinity binding) precluding any reasonable level of predictability and so the surprisingly high affinity binding of the In³⁺ and Ru³⁺ ions provides a useful tool in the detection and treatment of a range of conditions associated with gastrins, including non-amidated gastrins.

In one embodiment the indium and ruthenium are in the ⁺³ form i.e. In³⁺ and Ru³⁺.

In one embodiment, the indium and ruthenium may be radioisotopes of indium and ruthenium.

The metal may be administered as part of a solution containing the free ions or the metals may be a component of a compound or salt. The compound should be one that can dissociate or otherwise make the metal available for binding to the gastrin.

The compound containing the metal may be a simple, complex or organo-metallic salt or complex or chelate, polymorph, co-crystal or complex thereof, which is able to release the metal or otherwise make it available to occupy the ferric ion binding site or sites of non-amidated gastrins, and hence block their biological activity. An organometallic complex may include the trivalent metal ion bound to a convenient carrier, for example a cyclodextrin, or targeting molecule such as an antibody or the cation may be bound into a chelator or into an organic “wrapping” molecule such as the ruthenium derivatives of bipyridine and terpyridine. Suitable pharmaceutically acceptable simple salts may be derived from inorganic or mineral acids or alkalis, such as ammonia.

Simple complexes or salts of indium and/or ruthenium may be selected independently from oxides, carbonates, selenides, sulphates, aulphites, nitrates, nitrites, tribromides, trichlorides, acetates, citrates, malates, maleates, fumarates, succinicates, tartrates, salicylates, gallates, glycinates, glutamates, meslyates, picolinates and tosylates.

Larger complexes may include, but are not limited to, indium or ruthenium disalts such as hexaamineruthenium(II) chloride [Ru(NH₃)₆]Cl₂.

In another form the indium or ruthenium cation may be bound and/or attached to a variety of polymeric molecules such as PEG (polyethylene glycol) or a polylacate to lower the clearance rate from the body or provide a slow release of the cation from the substrate.

The condition associated with elevated concentrations of non-amidated gastrin may be any pathological condition in which the increased blood concentrations, rate of secretion or activity of Ggly are responsible for one or more symptoms of the condition. The condition may involve cell proliferation, cell migration, or acid secretion.

Preferably the condition is selected from the group consisting of gastrin-producing tumours, such as colorectal carcinomas; gastrinomas; islet cell carcinomas; ovarian tumours including stromal ovarian tumours; pituitary tumours; or from CCK-2 receptor expressing tumours, such as medullary thyroid carcinomas, astrocytomas, small cell lung cancers, meningiomas, endometrial and ovarian adenocarcinomas, breast carcinomas, gastrointestinal stromal tumours and gastro enteropancreatic tumours; conditions in which serum gastrins or their precursors are elevated, such as atrophic gastritis; G cell hyperplasia; pernicious anaemia; and renal failure; conditions affecting the gastrointestinal mucosa, such as ulcerative colitis.

Since non-amidated gastrin precursors are known to act as growth factors in the colonic mucosa, specific inhibitors of these gastrin precursors are useful for the treatment of disorders of gastrointestinal proliferation, such as ulcerative colitis and gastrointestinal cancers, as mentioned. In particular it is known that any prolonged elevation of gastrin concentrations increases the risk of colon cancer or pancreatic cancer. Thus the invention is applicable to the treatment or prevention of these conditions. The risk of colon cancer is also elevated in individuals on diets high in fat or meat, or in individuals with a family history of colon cancer such as familial adenomatous polyposis, who are therefore also suitable candidates for prophylactic treatment according to the invention.

Non-amidated gastrins also potentiate the stimulation of acid secretion by amidated gastrins, so specific inhibitors are also useful for the treatment of excessive acid production in patients with conditions such as gastrointestinal ulcers, gastro-oesophageal reflux, gastric carcinoid, or Zollinger-Ellison syndrome, including those being treated with proton pump inhibitors or H₂ blockers.

The invention represents a novel and unexpected method of blocking the biological actions of non-amidated gastrins. Occupation of the metal ion-binding site of non-amidated gastrins by indium or ruthenium ions prevents the binding of ferric ions, and so renders the peptide inactive. The major advantage of this approach is the specificity of inactivation. At the low concentrations of ruthenium or indium cations required to saturate the Ggly/Gamide binding site, there is likely to be little interference by the unbound metal ions with other biological processes.

The patient may be a mammal, particularly a human or a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as felids, canids, bovids, and ungulates.

Methods and pharmaceutical carriers for preparation of pharmaceutical compositions containing the metals are well known in the art, as set out in textbooks such as Remington's Pharmaceutical Sciences, 20th Edition (2000), Williams & Williams, USA.

The compounds and compositions of the invention may be administered by any suitable route or dose form, (including slow release dose forms) and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered. Bolus injection and IV infusion are just two approaches which may be useful.

The carrier or diluent, and other excipients, will depend on the route of administration or dose form, and again the person skilled in the art will readily be able to determine the most suitable formulation for each particular case.

The invention includes various pharmaceutical compositions useful for ameliorating disease. The pharmaceutical compositions according to one embodiment of the invention are prepared by bringing a metal or metal-containing compound of the invention or a derivative, complex, chelate or salt, copolymer thereof, or combinations of a compound of the invention and one or more other pharmaceutically-active agents, into a form suitable for administration to a subject using carriers, excipients and additives or auxiliaries.

Frequently used carriers or auxiliaries include but are not limited to magnesium carbonate, magnesium aluminium silicate, titanium dioxide, silicon dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobials, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals or may be given in an extended, depot or slow release format.

The pharmaceutical compositions according to the invention may be administered locally or, preferably, systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects, if any. Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, xanthan gum, gum tragacanth and gum acacia, magnesium silicate; dispersing or wetting agents, which may be (a) a naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also be a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

Metals of the invention may also be administered in the form of various simple or complex delivery systems, including but not limited to: liposome delivery systems, such as small unilamellar vesicles, large unilamellar vesicles, and multilamellar vesicles. Liposomes can be formed from a variety of phospholipids, such as cholesterol, stearylamine, or phosphatidylcholines; injectable microparticles; nanoparticles; matrix implants and device and/or bead coatings. These delivery systems may be devised to provide constant release, delayed release, pulsed release or sequential release concentrations of the metals of the invention. Carriers or materials may include; the cyclodextrins, poly(lactic-co-glycolic) acid, polyanhydrides, polylactides, poly-ortho-esters and hydrogels comprised of HPMC or other cellulose derivatives. Metals or compounds containing them may be attached to targeting molecules such as antibodies or receptor molecules.

The delivery form of the metal i.e. compound, complex salt etc. must be capable of hydrolysis or other dissociation to make the metal available to bind to the gastrin.

Dosage levels of the metals of the invention will usually be of the order of about 0.001 mg to about 250 mg per kilogram body weight, with a preferred dosage range between about 0.1 mg to about 10 mg per kilogram body weight per day (from about 0.1 g to about 3 g per average patient per day). The amount of active metal ion that may be combined with the carrier materials to produce a single dosage will vary, depending upon the host to be treated and the particular mode of administration. For example, a formulation intended for oral administration to humans may contain about 1 mg to 1 g of an active metal with an appropriate and convenient amount of carrier material, which may vary from about 5 to 95 percent of the total composition. Dosage unit forms will generally contain between from about 1 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other compounds to provide an operative combination or co-treatment. It is intended to include any chemically compatible combination of pharmaceutically-active agents, as long as the combination does not negatively impact upon the activity of the metals of this invention.

According to a second aspect of the invention there is provided a method of modulating the activity of a non-amidated gastrin including the step of binding a metal selected from indium and ruthenium to the non-amidated gastrin.

All of the comments made in relation to the first aspect apply equally well to the second aspect.

The modulation of the non-amidated gastrin will be a reduction in its normal biological activity. This is brought about by the ruthenium or indium blocking the ferric binding site, which ferric binding is required for normal activity.

According to a third aspect of the invention there is provided a method of treatment or prophylaxis of a condition in a patient associated with over-expression of a CCK receptor including the step of administering an effective amount of a metal selected from indium and ruthenium to the patient.

The metal is administered to the patient such that the metal binds to the ferric binding site of an amidated gastrin and, upon binding of the amidated gastrin to the CCK receptor, is internalised within a cell bearing the receptor.

In one embodiment the CCK receptor is a CCK-1 or CCK-2 receptor.

Preferably, the CCK receptor is CCK-2 receptor.

As discussed above in relation to Ggly, the present inventors have found that indium or ruthenium ions also bind to amidated gastrins with unexpectedly high affinities. In³⁺ has been found to have a K_{d} value of 6.5×10⁻¹⁵ M for the first iron binding site of Gamide versus 3.0×10⁻¹⁰ M for Fe³⁺. Ru³⁺ has been found to have a K_{d} value of 2.6×10⁻¹³ M for the first iron binding site of Gamide. Both metals therefore bind at affinities orders of magnitude higher than iron.

The benefit of the embodiments outlined herein is in taking advantage of the high specificity and affinity of the binding of Ru or In to the pentaglutamate sequence of Gamide itself. Further, the high affinity results in increased stability of the metal-gastrin complex.

The role of the CCK receptor has already been discussed in relation to tumours and so the invention allows the utilisation of ruthenium or indium cations as cytotoxic agents. Where the tumour/s are CCK-1 or CCK-2 receptor positive, treatment can be based on the knowledge that the CCK2R is internalised once Gastrin/Gamide has bound, together with any bound metal ion. If the metal ion is strongly and irreversibly bound, as has been proven to be the case with both indium and ruthenium ions, and is radiolabelled at sufficiently high specific activity, radiation damage to the contents of the cell can lead to cell death. In this “Trojan Horse” approach β-particle emitters, with an intermediate half-life, are effective. Binding of ruthenium or indium attached to Gastrin/Gamide and then the complex to CCK2R-expressing tumour cells will result in an increase in intracellular concentrations of the ions, which will substitute for Fe in many important Fe-containing proteins and enzymes, and thereby disrupt numerous intracellular processes essential for cell viability.

Radioactive isotopes of indium or ruthenium may be desirable in such treatment but are not essential. Ruthenium is immediately below iron in the periodic table, and would therefore be expected to be the closest available iron homologue. It is therefore expected that binding of Ru-Gamide to CCK2R-expressing tumour cells will result in an increase in intracellular concentrations of ruthenium, which will substitute for iron in many important iron-containing proteins and enzymes, and thereby disrupt numerous intracellular processes essential for cell viability. The same interference effect may be obtained from indium ions.

Therefore, in one embodiment, the indium and ruthenium are radioisotopes.

The condition treated may be any condition associated with the CCK receptor and includes those conditions, recited for the first aspect, that are associated with CCK receptor expression. The compositions, dosage and delivery forms may also be as described for the first aspect.

In one embodiment, the metal is not bound to a chelate group when it is brought into contact with the amidated gastrin. That is, the metal is not delivered as a component of a peptide-chelate conjugate or complex. Such chelate groups including DOTA, DTPA, HYNIC and tetraamine are discussed herein.

A fourth aspect of the invention resides in a method of delivering a metal selected from indium and ruthenium internally into a cell expressing a CCK receptor including the steps of contacting an amidated gastrin with the metal to allow it to bind thereto and allowing the amidated gastrin, with bound metal, to contact the CCK receptor and be internalised into the cell.

The fourth aspect may be carried out as described for the third aspect and all elements recited are considered therefore to be explicitly repeated for the fourth aspect.

According to a fifth aspect of the invention there is provided a method of forming a gastrin-metal complex including the step of contacting a gastrin with a metal selected from indium and ruthenium.

A sixth aspect of the invention resides in a complex comprising a gastrin and a metal selected from indium and ruthenium bound to the ferric binding site of the gastrin.

In one embodiment of the fifth and sixth aspects, the gastrin is an amidated gastrin.

In an alternative embodiment of the fifth and sixth aspects, the gastrin is a non-amidated gastrin.

The gastrin-ruthenium and gastrin-indium complexes of the invention have been found to be surprisingly stable due to their high affinity binding. The structures of the complexes of glycine-extended gastrin₁₇ with trivalent metal ions have been determined by X-ray absorption fine structure spectroscopy and are discussed in the experimental section. This high affinity also means that mild conditions can be used to bind the metal to the gastrin and so damage to the peptide is much less likely.

A seventh aspect of the invention resides in a method of diagnosing a CCK receptor positive cancer including the steps of:
(i) administering an amidated gastrin-metal radioisotope complex comprising an amidated gastrin complexed with a metal radioisotope selected from indium and ruthenium, to a patient;
(ii) allowing the amidated gastrin-metal radioisotope complex to become bound to the CCK receptor; and
(iii) detecting the presence of the metal radioisotope in the amidated gastrin-metal isotope complex,
to thereby diagnose the CCK receptor positive cancer.

An eighth aspect of the invention resides in a method of detecting a receptor for a non-amidated gastrin including the steps of:
(i) administering a non-amidated gastrin-metal radioisotope complex comprising a non-amidated gastrin complexed with a metal radioisotope selected from indium and ruthenium, to a patient;
(ii) allowing the non-amidated gastrin-metal radioisotope complex to become bound to the receptor; and
(iii) detecting the presence of the metal radioisotope in the non-amidated gastrin-metal radioisotope complex,
to thereby detect the non-amidated gastrin receptor.

In relation to the seventh and eighth aspects the detection of the CCK receptor positive cancer or receptor for a non-amidated gastrin is achieved via the high specificity and affinity of the binding of Ru or In to the gastrins.

The binding is to the pentaglutamate sequence of the gastrins, which is absent from minigastrin11 and so, in one embodiment of any one of the above eight aspects, the gastrin is not a minigastrin11 or minigastrin11 analogue. Advantageously, labelling occurs rapidly at room temperature, so that the peptide is unlikely to be damaged. In particular the high affinity results in increased stability of the metal-gastrin complex thereby avoiding one of the drawbacks of the prior art gastrins conjugated with metal chelate groups. It is expected that at least the Ru and In isotopes listed below will be well suited to location of gastrin-binding receptors and cells presenting them, including CCK2R-positive tumours, by Single Photon Emission Computed Tomography (SPECT) or Positron Emission Tomography (PET).

SPECT: ¹¹¹In-Gamide (γ emitter, t_{1/2}=67 h),
⁹⁷Ru-Gamide (γ emitter, t_{1/2}=65 h).
   PET: ¹⁰⁹In-Gamide (β emitter, t_{1/2}=4.3 h).

Therefore, where the tumours are CCK-1 or CCK-2 receptor positive, the complex of Gamide with a suitable radioactive isotope of ruthenium or indium may be used diagnostically to locate the original tumour or its metastases by PET or SPECT.

The radioisotope may therefore be selected from the group consisting of ¹⁰⁹In, ¹¹⁰In, ¹¹¹In, ⁹⁵Ru, ⁹⁷Ru, ¹⁰³Ru, ¹⁰⁵Ru and ¹⁰⁶Ru.

The data presented herein shows that radioactive isotopes of Ru or In could be directly complexed with amidated gastrins for use as CCK receptor probes in single-photon emission computed tomography (SPECT, ⁹⁷Ru, ¹¹¹In) and positron emission tomography (PET, ¹⁰⁹In). The use of a portable generator, as is known in the art, makes this approach more feasible. One advantage of this approach would be that oxidative damage to the peptide would be avoided, since complex formation proceeds rapidly at room temperature.

In contrast to the abundant structure—function information available for the CCK2R, the identities of the receptors for non-amidated gastrins such as progastrin and Ggly are still controversial. The recognition that Ggly and progastrin bind both Ru and In metals with high affinity provides novel tools for the identification of receptors for the non-amidated gastrins. It is expected that the Ru and In isotopes listed above are well suited to location, and hence subsequent identification, of tumours and other disease states expressing such receptors by SPECT or PET.

In one embodiment of any one or more of the above eight aspects, the gastrin, amidated or non-amidated, is one which comprises at least three of the five glutamate residues of the characteristic gastrin pentaglutamate sequence.

In one embodiment of any one or more of the above eight aspects, the gastrin, amidated or non-amidated, is one which comprises at least four of the five glutamate residues of the characteristic gastrin pentaglutamate sequence

In one embodiment of any one or more of the above eight aspects, the gastrin, amidated or non-amidated, is one which comprises a pentaglutamate sequence.

The above three statements regarding the pentaglutamate sequence, and the number of glutamate residues actually contained therein within the gastrin of interest, are particularly applicable to embodiments wherein a gastrin with bound radioactive metal is actually generated before its use, such as in administration to a patient. In this regard the gastrins employed in the complexes and methods of the invention are clearly differentiated from those gastrin derivatives, such as minigastrin11, which do not contain the pentaglutamate sequence.

It may not be essential that all five of the glutamate residues of the pentaglutamate sequence are present. For example Ggly with glu6 replaced by ala still binds 2 Fe with a similar affinity to the parent peptide. While certain residues have been observed in the present experimental as being particularly important it is believed that so long as three of the native glutamate residues of the characteristic pentaglutamate sequence are present in the gastrin employed in the complexes and methods of the present invention good results will be obtained. It is, however, preferred that at least four and, even more preferably, all five of the native glutamate residues are present in the form of the native pentaglutamate sequence.

In any one or more of the above eight aspects, the gastrin, amidated or non-amidated, is one which is chelate-free. That is, the gastrin is not conjugated, complexed or otherwise associated with a chelate or prosthetic group to which the ruthenium or indium binds or is intended to bind. Once again, this differentiates from prior art minigastrin11-DOTA and like approaches which bind and deliver the metals by a fundamentally different mechanism to that employed in the present invention.

In specific embodiments, therefore, the gastrin of any one or more of the above eight aspects does not comprise a DOTA, DTPA, HYNIC or tetraamine chelate group.

In certain embodiments, the gastrin of any one or more of the above eight aspects is not a gastrin selected from the group consisting of a minigastrin11 or minigastrin11 analogue, a demogastrin or demogastrin analogue, and a DTPA, DOTA or HYNIC conjugated minigastrin or demogastrin.

It is a significant advantage in that the present invention employs direct binding of the ruthenium or indium to the ‘unmodified’ gastrin and avoids the need for conjugation of any group added specifically to allow for metal binding. This means the gastrin itself is less likely to be damaged during preparation of the gastrin/metal complex and is likely to have significantly improved in vivo stability.

The invention will now be described in detail by way of reference only to the following non-limiting examples and drawings.

The various features and embodiments of the present invention, referred to in individual sections above apply, as appropriate, to other sections, mutatis mutandis. Consequently features specified in one section may be combined with features specified in other sections as appropriate.

The following examples are provided by way of illustration and are in no way limiting upon the scope of the invention.

### Experimental

### Materials and Methods

### Peptides and Metal Ions

Gamide and Ggly (88 and 93% pure, respectively) were purchased from Auspep (Clayton, Australia). The impurities consisted of water and salts. Solutions of metal ions (Aldrich, St. Louis, Mo.) were prepared in 10 mM HCl, and their concentrations determined by inductively coupled plasma-atomic emission spectroscopy at the National Measurement Institute (Pymble, Australia). Ru¹⁰⁶ (2 mCi/ml in 6M HCl) was purchased from Eckert & Ziegler Isotope Products (Valencia, Calif.).

### Absorption Spectroscopy

The 280 nm absorption of peptides (10 μM in 10 mM sodium acetate, pH 4.0, containing 100 mM NaCl and 0.005% Tween 20) in the presence of increasing concentrations of metal ions was measured against a buffer blank, in 1 ml quartz cuvettes thermostatted at 298 K, with a Cary 5 spectrophotometer (Varian, Mulgrave, Australia).

### X-Ray Absorption Sample Preparation, Spectroscopy and Analysis

Samples for X-ray absorption spectroscopy (XAS) were prepared with 1 mM peptide, 50 mM MOPS, 10% DMSO and 20% glycerol as a cryoprotectant. Metal stock solutions were prepared from the corresponding nitrate salt and titrated to a final concentration of 2 mM. Following data collection, samples containing 2 mM Ga or In were further titrated with 1 mM Fe for comparison. All samples were frozen in liquid N₂ prior to data collection. XAS measurements were conducted at the Stanford Synchrotron Radiation Laboratory with the SPEAR storage ring containing roughly 450 mA at 3.0 GeV, using the data acquisition program XAS Collect (29). Iron, gallium and indium K-edge data were collected on the structural molecular biology XAS beamline 7-3, operating with a 20-pole 2 Tesla wiggler source, and employing a Si(220) double-crystal monochromator. For Fe and Ga spectroscopy a downstream vertically collimating Rh-coated mirror was employed for harmonic rejection, such that the harmonic fell above the cut-off. Incident X-ray intensity was monitored using a nitrogen-filled ionization chamber and X-ray absorption was measured as the X-ray Kα fluorescence excitation spectrum using an array of 30 germanium detectors. X-ray fluorescence was collected through a Soller slit assembly, and intrinsic sample fluorescence registered by the detector was removed using filters of 6 or 9 absorption unit thickness (Mn for Fe, Zn for Ga, and Ag for In). During data collection, samples were maintained at a temperature of approximately 10 K using a liquid helium flow cryostat (Oxford Instruments). For each data set, 6-9 scans for each sample were accumulated, and the energy was calibrated by reference to the absorption of a reference foil of the same element, measured simultaneously with each scan (assuming a lowest energy inflection point of 7,111.3 for Fe, 10,368.2 for Ga, and 27,940.0 eV for In).

The EXAFS oscillations_{X}(k) were quantitatively analysed by curve-fitting using the EXAFSPAK suite of computer programs (http://www-ssrl.slac.stanford.edu/exafspak.html) as per the published method (George, 1996), using ab initio theoretical phase and amplitude functions calculated with FEFF v 8.20×5 (Rehr 1991). The energy thresholds of the extended X-ray absorption fine structure (EXAFS) oscillations (k=0 Å⁻¹) were assumed to be 7,130 for Fe, 10,385 for Ga, and 27,960 eV for In. Iron data was collected to a k-range of 14.2 Å⁻¹, Ga to k of 14.0 Å⁻¹, and In was collected to k of 16.2 Å⁻¹.

### Sep-Pak Purification

The Ru¹⁰⁶-Gamide complex was made by incubating 250 pmol of Ru¹⁰⁶ and 250 pmol of Gamide in 50 mM sodium acetate, pH 4.0, for 1 h at room temperature and was purified on reverse phase C18 Sep-Pak cartridges (Waters Corporation, Milford, Mass.). The Sep-Pak cartridges were first activated by passing through 10 mL of buffer A (100 mM Na acetate, pH 4), 10 mL of buffer B (100 mM Na acetate, pH 4, 50% acetonitrile) and 10 mL of buffer A. The reaction mixture was then passed through, and the cartridge was washed with 10 mL of buffer A to remove unbound Ru¹⁰⁶. The Ru¹⁰⁶-Gamide complex was eluted with buffer B, and the radioactivity in serial fractions (1 mL each) was detected with a β-counter (Packard, Meriden, Conn.). Fractions 1 and 2 contained Ru¹⁰⁶-Gamide complex and were either diluted in buffer A and filtered twice before being injected into the HPLC, or combined and dried in a Speed Vac (Savant, Hicksville, N.Y.) from 2 mL to around 100 μL before resuspension in binding buffer (see below) for the binding assay.

### HPLC Purification

The Ru¹⁰⁶-Gamide complex (discussed further below) was purified by anion exchange HPLC on a Protein-Pak Q 8HR (5×50 mm, Waters Corporation) at a flow rate of 1 mL/min with a gradient from 0 to 1 M NaCl in buffer A over 55 minutes. The radioactivity in 1 mL fractions was measured with a β-counter (Packard, Meriden, Conn.).

### Curve Fitting and Statistics

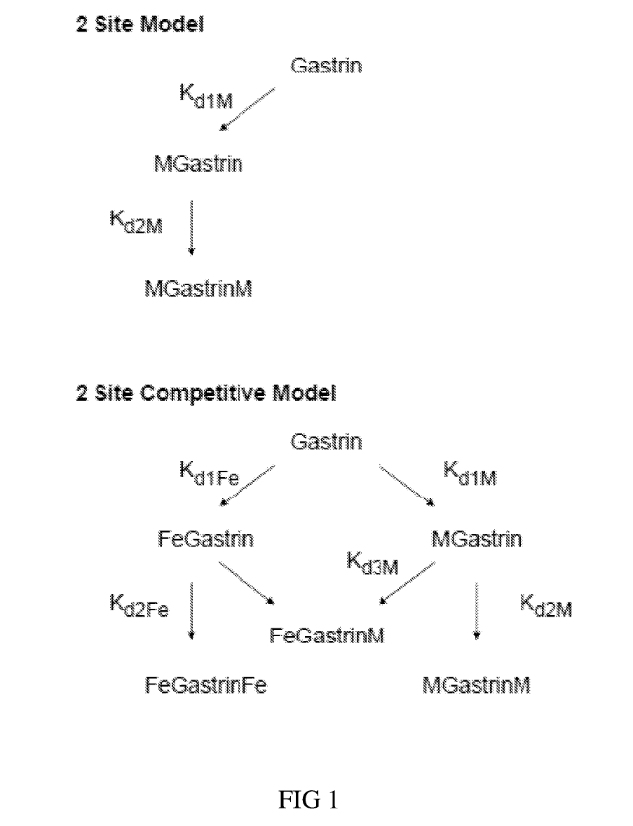

Data (expressed as means±S.E.M.) were fitted to one-site or two-site ordered models (as per FIG. 1) with the program BioEqs. The experimentally determined equilibrium constants and absorbance ratios given in Table 1 for the interaction of Gamide or Ggly with ferric ions were held constant while fitting the data for the interaction of other metal ions with Gamide or Ggly in the presence of ferric ions.

### Results

### Binding of Metal Ions to Gastrins

Iron Binding: The effect of addition of Fe³⁺ ions on the absorption spectrum and fluorescence of Gamide and Ggly at pH 4.0 has been reported previously (Baldwin, 2001). Fitting of a linear transformation of the fluorescence data was consistent with 2 binding sites with μM affinities, as indicated in FIG. 1. New absorption data sets were obtained, and fitted with the program Bioeqs as described in the Materials and Methods section. Reasonable fits were obtained with affinities of 3.0×10⁻¹⁰ and 8.5×10⁻¹¹M for Gamide and 5.7×10⁻⁹ and 7.0×10⁻⁹ M for Ggly (Table 1). As shown in FIG. 1, in the 2 site model gastrin binds two metal ions with dissociation constants K_{d1M} and K_{d2M}. In the 2 site competitive model gastrin binds two ferric ions with dissociation constants K_{d1Fe} and K_{d2Fe}, and two metal ions M to the same two sites with dissociation constants K_{d1M} and K_{d2M}. The dissociation constant K_{d3M} describes the formation of the mixed FeGastrinM complex.

### EXAFS Characterisation of Fe₂Ggly

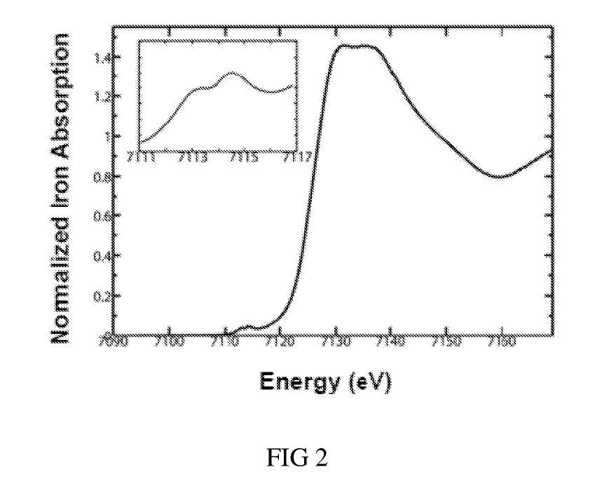

The XAS K-edge near edge spectrum of Fe^{III}₂Ggly (FIG. 2) demonstrates pre-edge peaks centred at 7,114 eV arising from 1 s→3 d (t_{2g}) and 1 s→3 d(e_{g}) transitions. The relatively large separation between these peaks (Δ=1.2 eV) arises from an elevation of the e_{g} levels, relative to the lower t_{2g} levels, and is indicative of low spin ferric iron. This large splitting also agrees with the expectation that the ferric ions are coordinated predominantly by the carboxylate donors of the glutamate side chains.

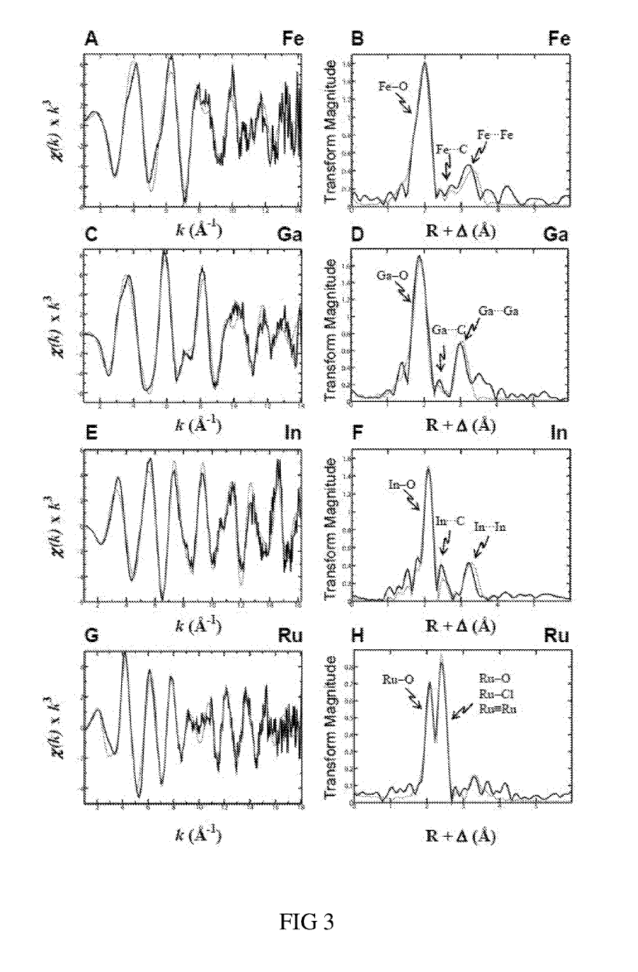

The Fe^{III}₂Ggly EXAFS data (FIG. 3A—wherein the K-edge extended X-ray absorption fine structure (EXAFS) spectra (A, C, E, G, solid black heavy lines) and the corresponding Fourier transforms (B, D, F, H) for the complexes of Ggly with Fe³⁺ ions (A, B), Ga³⁺ ions (C, D), In³⁺ ions (E, F) or Ru³⁺ ions (G, H) are shown together with the best fits (red/lighter and thinner lines) calculated using the single scattering path parameters listed in Table 2) is dominated by Fe—O backscattering interactions just below 2 Å, and an outer shell backscattering Fe . . . Fe interaction at ˜3.3 Å. The best fit to the data was obtained using single scattering paths, including 2 short Fe—O backscattering interactions at 1.90 Å, 4Fe—O at 2.03 Å, 1 Fe . . . C interaction at 2.57 Å, 2 Fe . . . C interactions at 2.96 Å and a single Fe . . . Fe interaction at 3.33 Å (Table 2). The structural parameters are reminiscent of the diferric non-heme iron binding proteins, such as methane monooxygenase and similar di-iron complexes, where the iron atoms are relatively close together and are bound by multiple carboxylates, including bridging carboxylates between the metal centres. Based on the number of coordinating ligands and longer range Fe . . . C scattering interactions, which appear prominent in the EXAFS data, the two ferric ions are predominantly bound by carboxylate donors with at least one bridging carboxylate. There is also a clear preference for inclusion of shorter Fe—O bond lengths (1.90 Å) in the fit, which may be indicative of bridging oxygen atoms, possibly as O²⁻ or OH⁻, although the internuclear separation is not particularly diagnostic in this case as mono-dentate carboxylate donors to Fe³⁺ can also fall close to this range of interatomic distances in similar complexes.

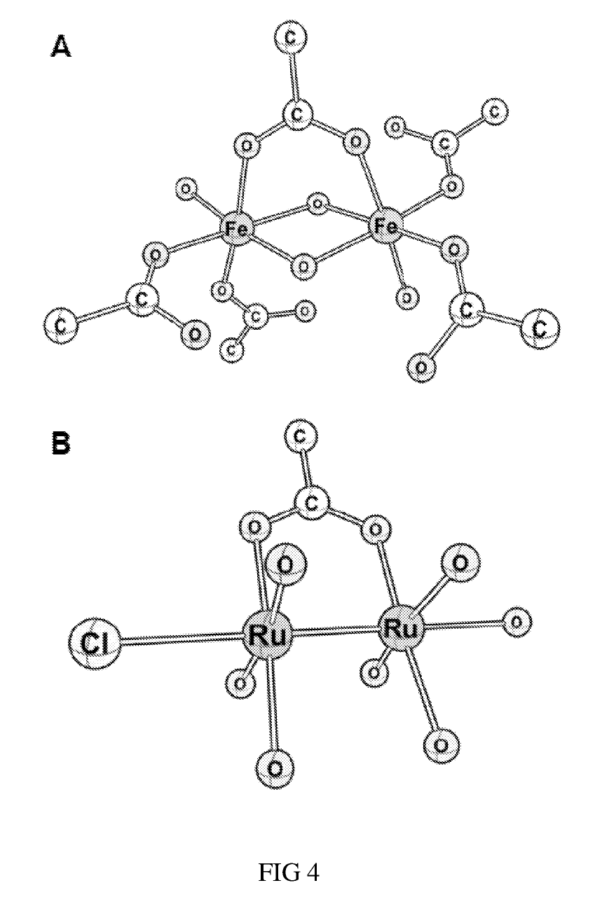

The EXAFS data was best fit by a single Fe . . . Fe scattering interaction. This observation indicates that Ggly binds Fe³⁺ in a di-iron coordination environment, as indicated in FIG. 4 which is a proposed model of the Fe^{III}₂Ggly structure based on the EXAFS data presented in FIG. 3 but which is also consistent with previous NMR and visible spectroscopic studies of Ggly and mutant peptides. The two Fe^{III} ions in the model of FIG. 4 are coordinated by the carboxylate side chains of glutamates 6, 7, 8, 9 and 10, with glutamate 7 acting as a ligand to both Fe^{III} ions. Two oxygens also act as bridges between the two Fe^{III} ions. The peptide backbone and non-coordinating side chains have been omitted from FIG. 4 for simplicity. The Ggly binding of Fe³⁺ in the di-iron coordination environment occurs without apparent recruitment of any additional ferric ions, as is otherwise often encountered in multinuclear small molecule crystal structures of iron-carboxylate complexes. The Fe . . . C interactions suggest that each iron centre interacts with at most two bridging carboxylates as well as at least one additional carboxylate that is not involved in a bridging interaction.

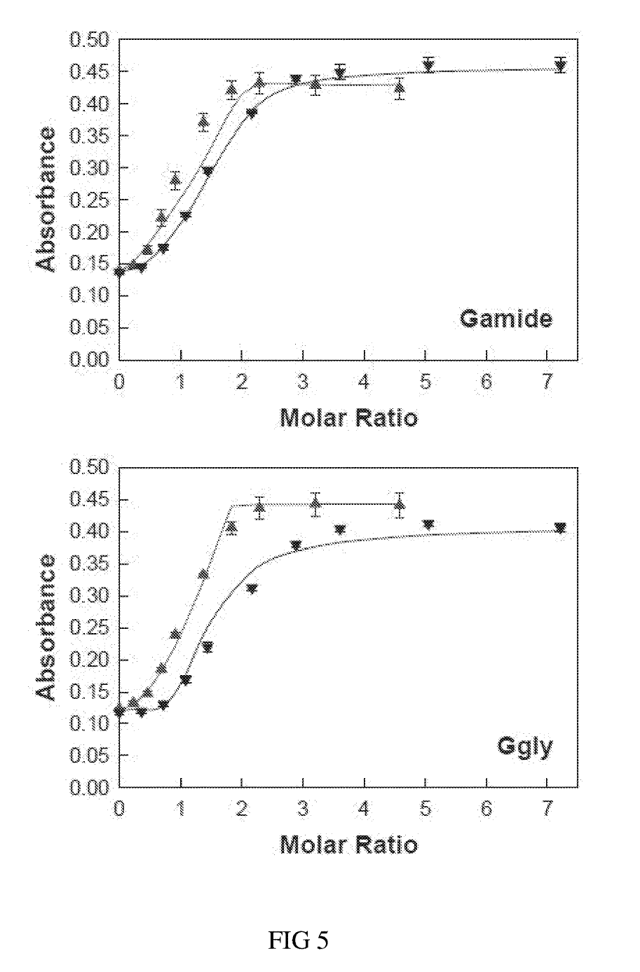

Gallium Binding: The addition of Ga³⁺ ions caused an increase in absorbance at 280 nm for both Gamide and Ggly at pH 4.0 as seen in FIG. 5. In FIG. 5 at pH 4.0 the addition of aliquots of ferric chloride ( [symbol] ) or gallium nitrate (▾) to 10 μM Gamide or Ggly in 10 mM Na⁺ acetate, 100 mM NaCl, 0.005% Tween 20 at 298 K is seen to have resulted in an increase in the absorption at 280 nm up to a molar ratio of 2.0 and fitting with the program Bioeqs yielded affinities for Ga³⁺ of 3.3×10⁻⁷ and 1.1×10⁻⁶M for Gamide and 1.7×10⁻⁸ and 2.3×10⁻⁶M for Ggly (Table 1). In FIG. 5 points are means of at least three separate experiments; bars represent the SEM. Lines represent the best fit to the two site model shown in FIG. 1 with the program BioEqs and the appropriate K_{d} and maximum absorbance values are given in Table 1.

### EXAFS Characterisation of Ga₂Ggly

Although the primary backscattering peak in the Ga^{III}₂Ggly EXAFS Fourier transform (FIG. 3D) appears more symmetric, significantly improved fits were obtained with the inclusion of two separate Ga—O backscattering interactions, two at 1.88 Å and three at 1.99 Å. The fact that inclusion of a third Ga—O backscatterer at 1.88 Å yielded little change in the fit suggested that the Ga³⁺ centres could be either 5- or 6-coordinate, although mixtures cannot be ruled out either. The EXAFS data (FIG. 3C) also clearly demonstrate a Ga . . . Ga backscattering interaction at 3.05 Å, and the results from the single scattering path model used for the Ga^{III}₂Ggly data (Table 2) agree well with the di-iron EXAFS model. The structural implication is that Ga³⁺, when coordinating to Ggly, appears to substitute for Fe³⁺ with minimal structural change in the local coordination environment of the di-nuclear coordination site.

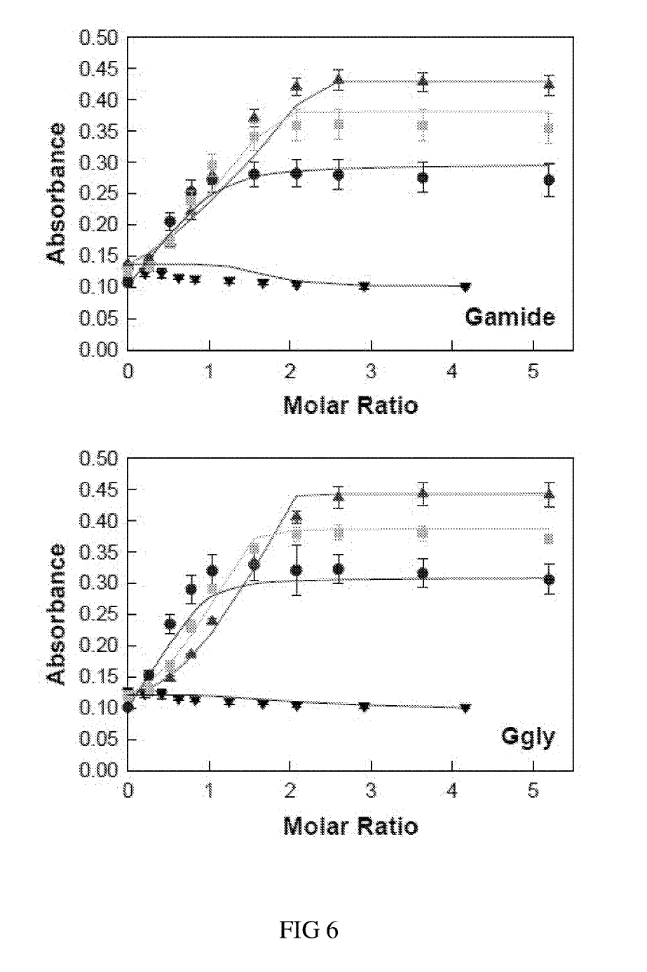

Indium Binding: The addition of In³⁺ ions caused little if any increase in absorbance at 280 nm for either Gamide or Ggly at pH 4.0 as is seen in FIG. 6. The graphical representation of FIG. 6 is obtained from addition of aliquots of indium nitrate (▾) to 10 μM Gamide or Ggly in buffer and resulted in little change in absorbance at 280 nm when compared to the changes seen on addition of aliquots of ferric chloride ( [symbol] ). However in the presence of 3.99 ( [symbol] ) or 39.85 μM ( [symbol] ) indium nitrate the changes in absorbance seen on addition of aliquots of ferric chloride were considerably different from the changes seen in the absence of indium nitrate. The points are means of at least three separate experiments; bars represent the SEM. The lines were constructed with the dissociation constants and maximum absorbance values (Table 1) obtained by fitting the data to the 2 site competitive model shown in FIG. 1 with the program BioEqs.

It is apparent then that, in the presence of 39.85 μM In³⁺ ions, the absorbance at 280 nm for both Gamide and Ggly on addition of Fe³⁺ ions increased more rapidly and approximated to the curve expected for single site binding, with the maximum absorbance reached near a molar ratio of 1. These observations are consistent with the hypothesis that an In³⁺ ion can bind to the first Fe³⁺ ion binding site with greater affinity than a Fe³⁺ ion, but without causing any change in absorbance. Indeed In³⁺ ions appear to compete for both Fe³⁺ ion binding sites, since the family of curves obtained at increasing concentrations of In³⁺ ions was reasonably well fitted with the program Bioeqs to the competitive two site model presented in FIG. 1. The best fit affinities of ions for the first Fe³⁺ ion binding site were substantially higher than for Fe³⁺ ions, with K_{d} values for In³⁺ of 6.5×10⁻¹⁵ and 2.1×10⁻¹³ M for Gamide and Ggly, respectively (Table 1).

### EXAFS Characterisation of In₂Ggly

The EXAFS Fourier transform (FIG. 3F) for In^{III}₂Ggly shows a shoulder on the shorter distance side of the primary backscattering peak centred at 2.1 Å, and the inclusion of a short In—O backscattering interaction significantly improved the fit. Truncating the k-range of the EXAFS data (FIG. 3E) to 14 Å⁻¹ confirmed that this apparent peak in the Fourier transform was reasonably well represented in the low k-range data, as would otherwise be expected for backscattering interactions with light atoms, such as oxygen, and was not attributable to noise or other artefacts. Overall the best fit to the EXAFS data was obtained by including a single short metal-O atom path at 1.98 Å as well as five equivalent In—O backscattering interactions at 2.13 Å. The In . . . In backscattering interaction was observed at 3.26 Å. The fact that the fit parameters for In^{III}₂Ggly agreed reasonably well with those used for the parent Fe^{III}₂Ggly complex suggested that, like Ga³⁺, In³⁺ coordinates to Ggly within a di-indium binding environment similar in structure to the Fe³⁺ complex.

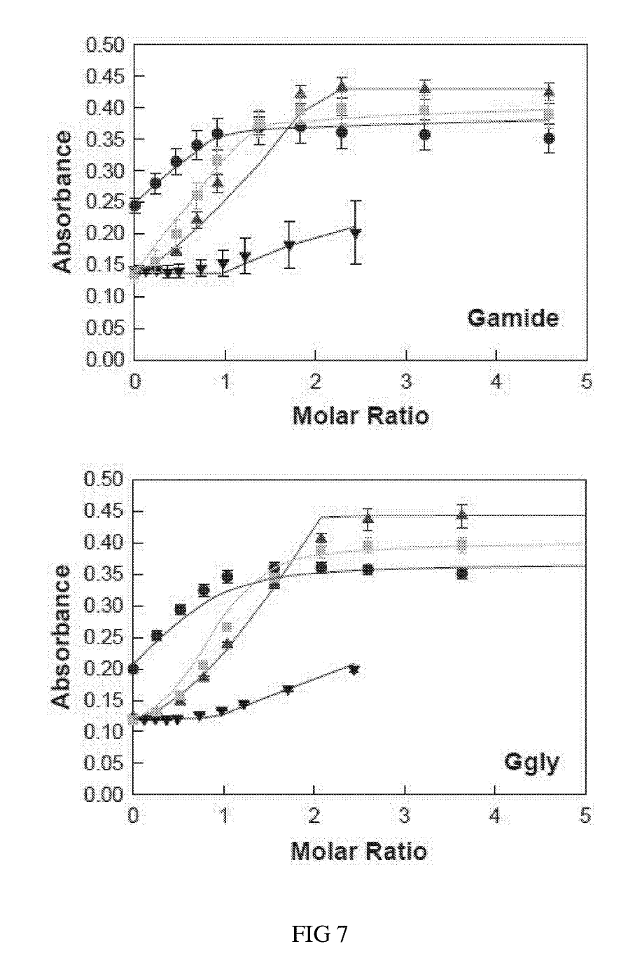

Ruthenium Binding: Similar families of curves were obtained when the binding experiments were repeated with Ru³⁺ ions instead of In³⁺ ions, as is represented in FIG. 7. The graphical representation of FIG. 7 is obtained from addition of aliquots of ruthenium chloride (▾) to 10 μM Gamide or Ggly in buffer which resulted in an increase in absorbance at 280 nm which was considerably less than the changes seen on addition of aliquots of ferric chloride ( [symbol] ). However in the presence of 5.30 ( [symbol] ) or 26.48 μM ( [symbol] ) ruthenium chloride the changes in absorbance seen on addition of aliquots of ferric chloride were considerably different from those seen in the absence of ruthenium chloride. The points are means from three separate experiments; bars represent the SEM. The lines were constructed with the dissociation constants and maximum absorbance values (Table 1) obtained by fitting the data to the 2 site competitive model shown in FIG. 1 with the program BioEqs.

The major difference between the ruthenium and indium observed results was that addition of Ru³⁺ ions itself caused a noticeable increase in absorbance at 280 nm for both Gamide and Ggly at pH 4.0. Nevertheless the family of curves obtained at increasing concentrations of Ru³⁺ ions was well fitted with the program Bioeqs to the competitive two site model presented in FIG. 1. The best fit affinities of Ru³⁺ ions for the first Fe³⁺ ion binding site were again substantially higher than for Fe³⁺ ions, with K_{d} values for Ru³⁺ of 2.6×10⁻¹³ and 5.3×10⁻¹⁵M for Gamide and Ggly, respectively (Table 1).

### EXAFS Characterisation of Ru₂Ggly

The EXAFS Fourier transform of the di-Ru³⁺ complex (FIG. 3H) is significantly different from those of the other complexes investigated and displays two intense backscattering peaks centred at ˜2.1 Å and ˜2.4 Å. The magnitude of the Fourier transform peaks in FIG. 3H is greatly diminished compared to those of the other complexes shown in FIGS. 3 B, D and F and is the result of significant cancellation between individual Ru scattering paths. The best fit to the data was obtained using a dinuclear Ru^{III} complex, containing a RuRu core, a bridging carboxylate and the remaining coordination completed with O-atoms and a single chloride bound to one of the Ru centres (FIG. 4B). Because the EXAFS (data FIG. 3G) experiment gives the superposition of all coordination environments about the Ru centres simultaneously, the fit parameters (Table 2) required fractional occupancy of CI as well as fractional occupancy of an O-atom at ˜2.4 Å in order to represent the non-equivalent coordination environments. This mixed dinuclear coordination environment also gave the maximal EXAFS cancellation represented by the experimental data. The short internuclear separation (2.4 Å) between the Ru centres is indicative of a direct metal-metal bond.

In summary, a systematic screen of a significant number of metal cations by ultraviolet absorption spectroscopy revealed that both Ggly and Gamide bound Ga³⁺, In³⁺ or Ru³⁺ in addition to Fe³⁺. With the trivalent Gallium cation, changes in the absorption of Gastrin in the presence of increasing concentrations of Ga³⁺ were fitted by a 2 site model with dissociation constants (K_{d}) of 0.33 and 1.1 μM or of 3.3×10⁻⁷ and 1.1×10⁻⁶M for Gastrin/Gamide and 1.7×10⁻⁸ and 2.3×10⁻⁶M for Ggly. Although the absorption of Gastrin did not change on addition of In³⁺ ions, the changes in absorbance on Fe³⁺ ion binding in the presence of Indium ions were fitted by a 2 site competitive model with K_{d} values for In³⁺ of 6.5×10⁻¹⁵ and 1.7×10⁻⁷M. Similar results were obtained with Ru³⁺ ions, with K_{d} values for Ru³⁺ of 2.6×10⁻¹³ and 1.2×10⁻⁵M (Table 1). The results demonstrate that both Gastrin/Gamide and Ggly selectively bind trivalent indium or ruthenium ions with significantly higher affinity than ferric or gallium ions.

TABLE 1
Binding of metal ions by Gamide and Ggly - The affinity of, and the
percentage absorbance change at 280 nm on, ferric ion binding to
Gamide or Ggly were determined by fitting the mean data obtained in
the absorbance experiments described in the appropriate FIGs discussed
previously with the program BioEqs.

| | | A₂₈₀ | | | K_{d3} | A₂₈₀ |
|---|---|---|---|---|---|---|
| | K_{d1} (M) | (%) | K_{d2} (M) | A₂₈₀ (%) | (M) | (%) |
| | Gamide | | | | | |
| Fe³⁺ | 3.0 × 10⁻¹⁰ | 100.0 | 8.5 × 10⁻¹¹ | 313.4 | | |
| Ga³⁺ | 3.3 × 10⁻⁷ | 100.0 | 1.1 × 10⁻⁶ | 335.8 | | |
| In³⁺ | 6.5 × 10⁻¹⁵ | 100.0 | 1.7 × 10⁻⁷ | 74.1 | 4.0 × 10⁻⁹ | 217.7 |
| Ru³⁺ | 2.6 × 10⁻¹³ | 100.0 | 1.2 × 10⁻⁵ | 178.6 | 1.7 × 10⁻⁸ | 264.9 |
| | Ggly | | | | | |
| Fe³⁺ | 5.7 × 10⁻⁹ | 100.0 | 7.0 × 10⁻⁹ | 365.9 | | |
| Ga³⁺ | 1.7 × 10⁻⁸ | 100.0 | 2.3 × 10⁻⁶ | 340.2 | | |
| In³⁺ | 2.1 × 10⁻¹³ | 100.0 | 1.4 × 10⁻⁵ | 72.8 | 9.6 × 10⁻⁸ | 255.6 |
| Ru³⁺ | 5.3 × 10⁻¹⁵ | 100.0 | 3.6 × 10⁻⁴ | 1714.8 | 1.2 × 10⁻⁶ | 304.1 |

TABLE 2
EXAFS curve fitting results - Coordination numbers, N, interatomic
distances R (Å), Debye-Waller factors σ² (Å²), and
threshold energy shift ΔE₀ (eV), were derived from EXAFS
curve-fitting. The fit error parameter F is defined as
F = {square root over (Σk⁶(χ(k)_{calc} − χ(k)ₑₓₚₜ)²/Σk⁶ χ(k)ₑₓₚₜ²)}, with the summation being
over data points included in the fit.

| Path | N | R | σ² | ΔE₀ | F |
|---|---|---|---|---|---|
| Fe^{III}₂Ggly | | | | | |
| Fe—O | 2 | 1.902(6) | 0.0025 | −6.7(6) | 41.29 |
| Fe—O | 4 | 2.029(4) | 0.0025 | | |
| Fe . . . C | 1 | 2.57(2) | 0.0045 | | |
| Fe . . . C | 2 | 2.96(2) | 0.0045 | | |
| Fe . . . Fe | 1 | 3.330(6) | 0.0035 | | |
| Ga^{III}₂Ggly | | | | | |
| Ga—O | 2 | 1.877(4) | 0.0025 | −7.8(6) | 31.43 |
| Ga—O | 3 | 1.985(3) | 0.0025 | | |
| Ga . . . C | 1 | 2.60(2) | 0.0045 | | |
| Ga . . . C | 2 | 3.02(1) | 0.0045 | | |
| Ga . . . Ga | 1 | 3.046(4) | 0.0035 | | |
| In^{III}₂Ggly | | | | | |
| In—O | 1 | 1.979(6) | 0.0025 | −14.6(5) | 32.17 |
| In—O | 5 | 2.132(2) | 0.00225 | | |
| In . . . C | 2 | 2.635(7) | 0.0045 | | |
| In . . . C | 2 | 3.09(2) | 0.0045 | | |
| In . . . In | 1 | 3.266(3) | 0.0035 | | |
| Ru^{III}₂Ggly | | | | | |
| Ru—O | 2 | 2.069(4) | 0.0032 | +3.6(5) | 38.84 |
| Ru—O | 2 | 2.173(6) | 0.0032 | | |
| Ru—Cl | 0.5 | 2.51(1) | 0.0033 | | |
| Ru≡Ru | 1 | 2.418(4) | 0.0040 | | |
| Ru—O | 0.5 | 2.42(5) | 0.0053 | | |
| Ru . . . O . . . Ru | 2 | 3.30(1) | 0.0054 | | |

Values in parentheses are the estimated standard deviations obtained from the diagonal elements of the covariance matrix; these are precisions and are distinct from the accuracies which are expected to be larger (ca ±0.02 Å for R, and ±20% for N and σ²), although relative accuracies (e.g. comparing two different Fe—O bond-lengths) will be more similar to the precisions.

### Purification and Stability of the Ru¹⁰⁶-Gamide Complex

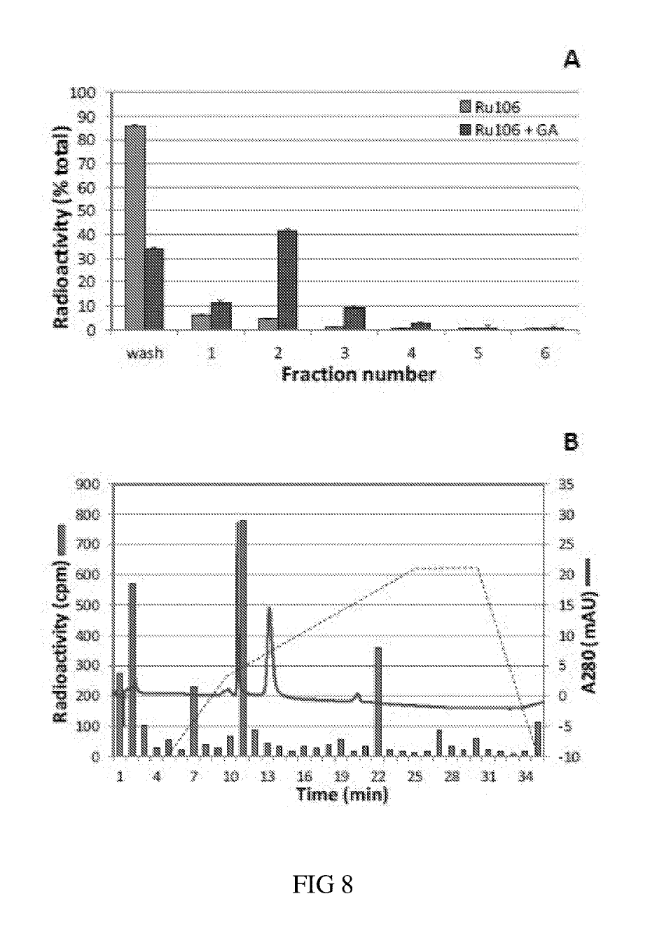

The first step in the purification of the Ru¹⁰⁶-Gamide complex utilised C18 SepPak cartridges. Unbound Ru¹⁰⁶ passed through the column in the initial flow through, while the Ru¹⁰⁶-Gamide complex eluted in fractions 1 and 2 (FIG. 8A). To purify the Ru¹⁰⁶-Gamide complex further, fractions 1 and 2 from the C18 SepPak cartridge were pooled and subjected to anion-exchange HPLC (FIG. 8B). The radioactivity found in fraction 11 clearly matches with the Gamide absorption peak. Similar results were obtained with the Ru¹⁰⁶-Ggly complex (data not shown). Importantly, the Ru¹⁰⁶⁻amidated Gastrin complex is very stable with a half-life at neutral pH of 16.8±3.2 days.

In detail, for the data represented in FIG. 8A the Ru¹⁰⁶-Gamide complex (darker shaded bars which are rightmost in each set of two) in 100 mM sodium acetate pH 4 was separated from unbound Ru¹⁰⁶ by binding to a C18 Sep-Pak cartridge, followed by washing with 10 mL 100 mM sodium acetate pH 4 and elution with 1 mL aliquots of 100 mM sodium acetate pH 4 containing 50% acetonitrile. When Ru¹⁰⁶ (lighter shaded bars which are leftmost in each set of two bars) alone was applied to the cartridge, most of the radioactivity was found in the initial wash. For FIG. 8B the Ru¹⁰⁶-Gamide complex was further purified by anion exchange HPLC using the indicated linear gradient from 0 to 1M NaCl in 10 mM sodium acetate pH 4 over 55 min. The radioactivity in each 1 ml fraction is indicated by the vertical bars.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this patent specification is intended to embrace all alternatives, modifications and variations of the present invention that have been discussed herein, and other embodiments that fall within the spirit and scope of the above described invention.

In the claims which follow and in the preceding description of the invention, except where the context clearly requires otherwise due to express language or necessary implication, the word “comprise”, or variations thereof including “comprises” or “comprising”, is used in an inclusive sense, that is, to specify the presence of the stated integers but without precluding the presence or addition of further integers in one or more embodiments of the invention.

### BIBLIOGRAPHY

Aly A, Shulkes A, & Baldwin G S (2004) Gastrins, cholecystokinins and gastrointestinal cancer. Biochim Biophys Acta 1704:1-10.
Baldwin G S, Curtain C C, & Sawyer W H (2001) Selective, high-affinity binding of ferric ions by glycine-extended gastrin(17). Biochemistry 40:10741-10746.
BALDWIN, G. S. (2004) Properties of the complex between recombinant human progastrin and ferric ions. The Protein Journal. 23: 65-70.
Dockray G J, Varro A, Dimaline R, & Wang T (2001) The gastrins: their production and biological activities. Annu Rev Physiol 63:119-139.
Fani M, Maecke, H. R., Okarvi S. M. (2012) Radiolabeled Peptides: Valuable Tools for the Detection and Treatment of Cancer. Theranostics; 2(5):481-501.
Ferrand A & Wang T C (2006) Gastrin and cancer: a review. Cancer Lett 238:15-29.
George G N, Garrett, R. M., Prince, R. C., Rajagopalan, K. V. (1996) The Molybdenum Site of Sulfite Oxidase: A Comparison of Wild-Type and the Cysteine 207 to Serine Mutant Using X-ray Absorption Spectroscopy. Journal of the American Chemical Society 118:8588-8592.
Goetze J P, et al. (2013) Characterization of gastrins and their receptor in solid human gastric adenocarcinomas. Scand J Gastroenterol 48:688-695.
Hollande F, et al. (1997) Glycine-extended gastrin acts as an autocrine growth factor in a nontransformed colon cell line. Gastroenterology 113:1576-1588.
Koh T J, et al. (1999) Overexpression of glycine-extended gastrin in transgenic mice results in increased colonic proliferation. J Clin Invest 103:1119-1126.
Pagliocca A, et al. (2002) Stimulation of the gastrin-cholecystokinin(B) receptor promotes branching morphogenesis in gastric AGS cells. Am J Physiol Gastrointest Liver Physiol 283:G292-299.
Rehr J J, Mustre de Leon, J., Zabinsky, S. I., Albers, R. C. (1991) Theoretical x-ray absorption fine structure standards. Journal of the American Chemical Society 113:5135-5140.
Reubi J C, Schaer J C, & Waser B (1997) Cholecystokinin(CCK)-A and CCK-B/gastrin receptors in human tumors. Cancer Res 57:1377-1386.
Roosenburg S, Laverman P, van Delft F L, & Boerman O C (2011) Radiolabeled CCK/gastrin peptides for imaging and therapy of CCK2 receptor-expressing tumors. Amino Acids 41:1049-1058.
von Guggenberg E, et al. (2012) Preclinical evaluation of radiolabeled DOTA-derivatized cyclic minigastrin analogs for targeting cholecystokinin receptor expressing malignancies. Mol Imaging Biol 14:366-375.
Wang T C, et al. (1996) Processing and proliferative effects of human progastrin in transgenic mice. J Clin Invest 98:1918-1929.

1. A method of treatment or prophylaxis of a condition in a patient associated with elevated concentrations of a non-amidated gastrin including the step of administering an effective amount of a metal selected from indium and ruthenium to the patient.

2. A method of modulating the activity of a non-amidated gastrin including the step of binding a metal selected from indium and ruthenium to the non-amidated gastrin.

3. The method of claim 1, wherein the metal is administered to the patient such that the metal binds to the ferric binding site of the non-amidated gastrin.

4. The method of claim 1, wherein the indium and ruthenium are in the In³⁺ and Ru³⁺ form.

5. The method of claim 1, wherein the indium and ruthenium are radioisotopes of indium and ruthenium.

6. The method of claim 1, wherein the metal is part of a solution containing the free ions, or the metal is a component of a compound or salt.

7. The method of claim 1, wherein the metal is not bound to a chelate group when it is brought into contact with the non-amidated gastrin.

8. The method of claim 1, wherein the condition is selected from the group consisting of a colorectal carcinoma, a gastrinoma, an islet cell carcinoma, an ovarian tumor, a pituitary tumor, a medullary thyroid carcinoma, an astrocytoma, a small cell lung cancer, a meningioma, an endometrial and ovarian adenocarcinoma, a breast carcinoma, a gastrointestinal stromal tumor, a gastro enteropancreatic tumor, atrophic gastritis, G cell hyperplasia, pernicious anaemia, renal failure, ulcerative colitis, a gastrointestinal ulcer, gastro-oesophageal reflux, gastric carcinoid, and Zollinger-Ellison syndrome.

9. The method of claim 1, wherein the patient is a mammal.

10. The method of claim 2, wherein modulation of the non-amidated gastrin is a reduction in its normal biological activity.

11. A method of treatment or prophylaxis of a condition in a patient associated with over-expression of a CCK receptor including the step of administering an effective amount of a metal selected from indium and ruthenium to the patient.

12. (canceled)

13. (canceled)

14. (canceled)

15. (canceled)

16. (canceled)

17. (canceled)

18. A complex comprising a gastrin and a metal selected from indium and ruthenium bound or complexed to the ferric binding site of the gastrin.

19. The complex of claim 18, wherein the gastrin is an amidated gastrin or a non-amidated gastrin.

20. The complex of claim 18, wherein the metal is not bound to a chelate group when it is brought into contact with the gastrin.

21. (canceled)

22. (canceled)

23. (canceled)

24. (canceled)

25. (canceled)

26. (canceled)

27. (canceled)

28. (canceled)

29. The method of claim 2, wherein the indium and ruthenium are in the In³⁺ and Ru³⁺ form.

30. The method of claim 2, wherein the indium and ruthenium are radioisotopes of indium and ruthenium.

31. The method of claim 2, wherein the metal is part of a solution containing the free ions or the metal is a component of a compound or salt.

32. The method of claim 2, wherein the metal is not bound to a chelate group when it is brought into contact with the non-amidated gastrin.
